(19) United States
(12) Patent Application Publication
(10) Pub. No.: US 2012/0083798 A1
(43) Pub. Date: Apr. 5, 2012

(54) INFLATABLE BELT TO ASSIST LABOR AND/OR CHILDBIRTH

(76) Inventor: Pierfrancesco Belli, Firenze (IT)

(21) Appl. No.: 13/321,244

(22) PCT Filed: May 19, 2010
(86) PCT No.: PCT/IB10/01164

§ 371 Date: Dec. 16, 2011

(30) Foreign Application Priority Data
May 22, 2009 (IT) FI2009A000115
May 22, 2009 (IT) FI2009A000116
May 22, 2009 (IT) FI2009U000043

(51) Int. Cl. A61B 17/42

(52) U.S. Cl. 606/121

(57) ABSTRACT

An equipped modular inflatable belt as aid to labour and delivery, whether at home or in hospital, which can be used even at a distance and remotely, comprising a supporting band (1) and at least one inflatable chamber applied to said band provided with ducts for connection to a supply of an inflating fluid, said belt comprising an electropneumatic apparatus applied to the band (1).

### STATE OF THE ART

The present invention regards an inflatable belt, which can be used as an aid during labour and delivery whether the parturient is confined to hospital or she is at her own home, and which can be used both remotely and at a distance.

As is known, during the phase of labour the uterus starts to contract, and, with the passage of time, said contractions become more frequent and intense. Frequently, however, it happens that said contractions, for a series of reasons in part known and in part still unknown, do not prove sufficient to bring about delivery within a short and/or acceptable time. It is likewise known that it is not possible to put off delivery beyond a maximum time: in fact, if there is a delay in delivery, there could be complications for the health of the mother and of the child.

Consequently, when the maximum time for awaiting parturition is approached, the health staff puts into practice whatever it is possible to do to enable rapid expulsion of the foetus from the uterus.

In particular, if the conditions so permit, the so-called Kristeller manoeuvre is performed, a practice that is widely adopted throughout the world.

Said manoeuvre consists, during the second phase of labour, in exerting a series of thrusts using hands, fists, forearms and knees on the part of the health staff in order to speed up delivery of the foetus from the uterus.

It is also known to operators of the sector that said manoeuvre is extremely violent and potentially dangerous and that it can cause permanent damage to the mother and foetus, even leading to the death of both in so far as even violent thrusts are impressed on the bottom of the uterus without having any possibility of effective control over the amount of force exerted.

To solve this problem there has been proposed the equipment described in the patent application No. FI2003A0588, which discloses the use of a band that can be inflated in a controlled way, said band being applied to the abdomen of the mother and inflated at the right moment and with the necessary force, assessed by the physician and/or by the health staff on the basis of bioelectrical signals of the mother and of the foetus detected by means of appropriate sensors.

The solution now described affords the considerable advantage of enabling both an action of accompaniment of the contraction during labour and an action of thrust during delivery, said action being controlled and performed in complete safety for both the mother and the foetus, thus avoiding the risks linked to the Kristeller manoeuvre.

This known solution presents, however, the drawback of requiring a stable connection to the pneumatic apparatus that carries out inflation of the belt, which prevents the parturient from being able to move.

A second drawback is represented by the fact that the solutions of a known type do not enable control of the pressure exerted in each point of the area of inflation.

Consequently, it is impossible to act only in a portion of the area of inflation when it is necessary or desirable to differentiate the thrust on the abdomen of the parturient.

For example, an operation of this type is desirable to stimulate a correct positioning of the foetus both during labour and prior to expulsion of the foetus from the uterus.

### PURPOSE OF THE INVENTION

The purpose of the present invention is to overcome the drawbacks of the solutions already known and propose an equipped modular inflatable belt that is able to exert different pressures in distinct areas of application of the belt, according to the clinical requirements at a given moment, and simultaneously to monitor the bioelectrical parameters of the mother and foetus.

### SUMMARY OF THE INVENTION

The above purpose has been achieved by providing an inflatable belt according to at least one of the annexed claims.

A first advantage lies in the possibility for the parturient to continue to wear the belt and move freely during labour also in the ward, or in the delivery room, or at her own home, in her own bed, monitoring being in any case maintained both for the mother and for the foetus, and in the possibility, if need be, for the parturient to be taken to the delivery room with the band and the sensors for monitoring the bioelectrical signals of the mother and foetus ready for use, the apparatus being usable even at a distance and remotely.

A further advantage lies in the possibility of applying the pressure in a differentiated way in different areas of the belt, without having to reposition the belt.

In particular, the belt according to the invention, in addition to exerting an active thrust, can be used in a passive way, i.e., for delimiting areas of confinement or constriction of the abdomen of the parturient in order to reduce the space available in the upper part and thus favour positioning of the foetus within the uterus, during labour, simply by varying the inflated part of the belt progressively.

Yet a further advantage lies in the possibility of selecting a direction of thrust for positioning the foetus with respect to the neck of the uterus and contributing to a correct positioning of the foetus during labour and for expulsion.

According to a further aspect, the invention regards an electromedical apparatus for assistance to labour and delivery, integrated in use with an inflatable belt, which is intended to overcome the drawback of apparatuses of a known type whereby the operators are not able to obtain a complete picture of labour/delivery, i.e., as regards the effective clinical conditions both of the mother and of the foetus, except by using additional dedicated instrumentation. As these conditions vary, the decisions to be taken during assistance to labour/delivery can in fact be very different and require very different therapies, as well as the presence of various skills and/or distinct instrumentation, which may normally not be present in the delivery room.

It is consequently desirable to have available a biomedical instrument for assistance to labour and delivery that will ensure complete and flexible monitoring of the mother and the foetus in the different possible circumstances of conditions of labour and delivery and that will enable the medical staff to acquire all the necessary information.

It is moreover desirable to have available a biomedical apparatus for integrated monitoring with a pneumatic inflating assembly that, by the action of gentle inflation of a purposely designed belt, applied to the parturient by the health staff, can act in accompanying each contraction during the phase of labour, enabling, on the one hand, the head of the foetus to be kept in the correct position where it exerts pressure on the neck of the uterus and, on the other, the subsequent contraction to be favoured by being accompanied gently, so that delivery is accomplished in complete safety for mother and foetus alike. At the same time, when the moment of delivery arrives, a thrust of expulsion is exerted, which is controlled throughout by means of the pneumatic belt itself applied to the abdomen of the mother, once again in complete safety for mother and foetus alike, via an action that can be monitored constantly both during labour and during delivery.

The aim of the present invention is to overcome the drawbacks of the solutions already known and to propose an electromedical apparatus that is able to provide, both for the mother and for the foetus, monitoring of the most significant vital and/or biological parameters, as well as to make immediately available to the physician and to the health staff the most relevant clinical information according to the effective circumstances that arise during labour and delivery.

A further purpose of the invention is to propose an integrated apparatus comprising a system for monitoring the mother and foetus during labour and delivery, as well as a pneumatic system for managing an inflatable band or belt, that are to be used for exerting both a thrust for accompanying contraction during labour and an additional expulsive thrust controlled on the basis of the values of the parameters monitored and are usable both at a distance and remotely.

Yet a further purpose is to provide an apparatus for assistance to delivery, the main characteristic of which is its mobility, as well as the possibility of supervising all the functions without any need for it to be connected physically to an external PC for use thereof, so that it can accompany the parturient right from start of labour, whether she be at home or in hospital, at her bedside up to her entry into the delivery room so as to afford thorough-going traceability not only of the actions of active aid, by means of the belt, but also of the physiological parameters of the mother and foetus, said apparatus being usable both at a distance and remotely.

### LIST OF THE DRAWINGS

The above and further advantages will be better understood by any person skilled in the branch from the ensuing description and the annexed plates of drawings, which are provided purely by way of non-limiting example and in which:

FIG. 1 shows a belt according to the invention;

FIGS. 2-4 are schematic illustrations of three different arrangements of the areas of inflation along the development of the belt;

FIG. 5 shows a belt according to the invention with separate inflating chambers;

FIG. 6 shows a belt according to the invention with separate inflating sectors of a single chamber;

FIG. 7 shows an apparatus for assistance to delivery comprising an inflatable belt;

FIG. 8 shows a possible diagram of electropneumatic components applied to the belt;

FIG. 9 shows in perspective view a belt according to the invention;

FIG. 10 is a schematic illustration of a possible pneumatic circuit that can be used with the invention;

FIGS. 11-13 show, respectively, a front view of an apparatus according to the invention, a side view, with the connections to the electric-power supply highlighted, and a side view, with the functional connections between the components of the apparatus highlighted;

FIG. 14 shows a possible diagram of the pressure device;

FIG. 15 shows a possible diagram of the control electronics of the pressure device;

FIG. 16 shows a possible diagram of the monitoring device;

FIGS. 17a and 17b show a possible diagram of an intermediate circuit for acquisition from non-bioelectrical sensors, respectively ultrasound sensors (FIG. 17a) and a tocographic sensor and a saturometer (FIG. 17b);

FIGS. 18a and 18b, show, respectively, a possible diagram of a single-chamber pneumatic belt and a multiple-chamber pneumatic belt, which can be used with the apparatus according to the invention;

FIG. 19 is a schematic illustration of a preferred embodiment of an apparatus according to the invention; and

FIG. 20 shows a possible flowchart of the electronic card of the apparatus of FIG. 19.

### DETAILED DESCRIPTION

Described with reference to the attached drawings is an inflatable belt 1 that can be used as an aid to delivery, comprising a supporting sheath 40, preferably having an ergonomic shape and being made of biocompatible material.

According to the invention, the belt 1 is provided with at least one inflatable chamber made for example of PVC cloth and supported by the sheath 40, with one or more areas of inflation 4. The belt 1 can moreover advantageously be wrapped around the abdomen of the parturient by closing means 9, set at the ends of the sheath 40 and of a lumbar support 29, preferably of the removable type, for example by hooks or Velcro®.

Advantageously, the use of Velcro or of a graduated succession of hooks enables even manual adjustment of the pressure and sliding and positioning of the belt on the abdomen.

Preferably, the sheath 40, (which has an opening for insertion of the inflating chamber or chambers) or the belt itself, is made of a light semirigid material, as outermost structure, or else, has inside it an insert made of semirigid material in order to strengthen the structure and to apply the pneumatic force principally towards the abdomen of the parturient and to enable wearing and tightening thereof with greater ease, it being possible to house in a stable way the mechanical and electrical mechanical parts described hereinafter in positions that are not troublesome for the parturient.

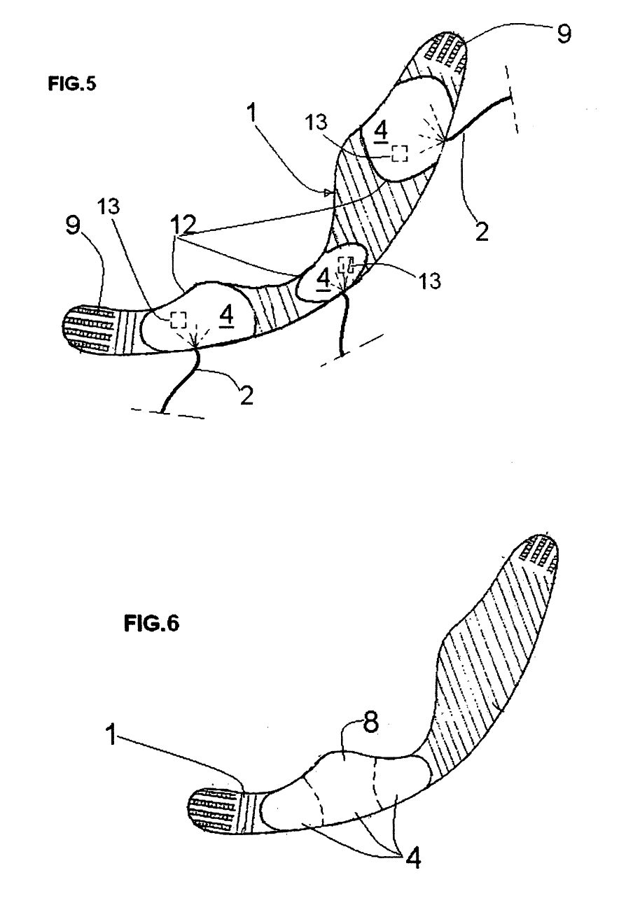

In various embodiments of the invention, a number of areas of inflation 4 may be envisaged, which can be inflated individually and are constituted by separate inflatable chambers 12 (FIG. 5) or else are obtained from separate sectors of one and the same inflatable chamber 8 (FIG. 6).

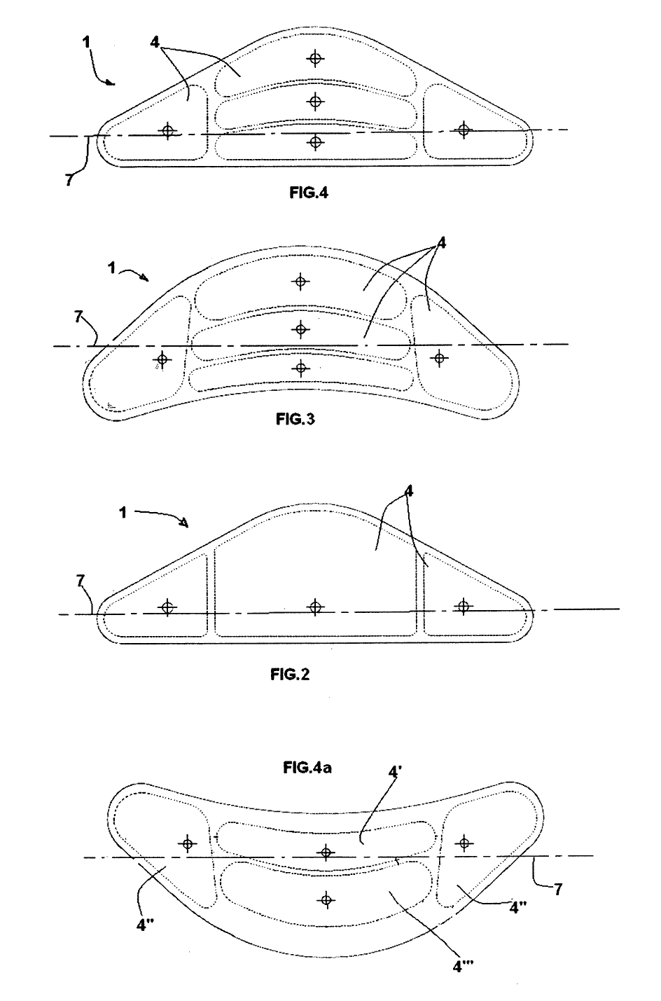

Furthermore, according to the requirements that each delivery can present, the areas of inflation 4 are set along a longitudinal axis 7 of wrapping of the belt around the abdomen or transversely with respect thereto, with a linear or more or less accentuated curved shape, as illustrated schematically in FIGS. 2-4. Illustrated in FIG. 4a is a conformation of the belt shaped like an arch concave downwards that has proven ergonomically most suitable. In this embodiment, the areas of inflation 4′, 4″, 4′″ can in fact be activated subsequently to facilitate displacement of the foetus in the uterus by varying the area of active pressure (action of thrust) or passive pressure (action of containment) exerted by the belt on the abdomen of the parturient.

With reference in particular to FIGS. 2-4, it may be noted that according to a further advantageous aspect of the invention, the present disclosure proposes an inflatable belt of modular conformation, which is able, that is, even separately from a control apparatus applied thereto in a unitary way, to exert different pressures in distinct areas of application of the belt according to the clinical requirements at a given moment, even without having to reposition the belt.

In particular, the belt according to the invention, in addition to exerting an active thrust, can be used in a passive way, i.e., for delimiting areas of confinement or constriction of the abdomen of the parturient in order to reduce the space available in the upper part and/or laterally and thus favour natural displacement of the foetus within the uterus, above all during labour, simply by varying progressively the inflated part of the belt. The use of the three pneumatic chambers, which can be inflated separately in a parametric way, enables a further control on the direction of the pressure exerted in order to enable a gradual thrust on the bottom of the uterus, both from above downwards and from right to left, or vice versa (FIG. 2).

Said belt consequently represents the form ergonomically most adaptable to the different physical conditions of parturients (weight-to-height ratio, weeks of pregnancy/parity).

Yet a further advantage lies in the possibility of selecting a direction of thrust for positioning the foetus with respect to the uterus and contributing to a correct positioning during labour and for expulsion.

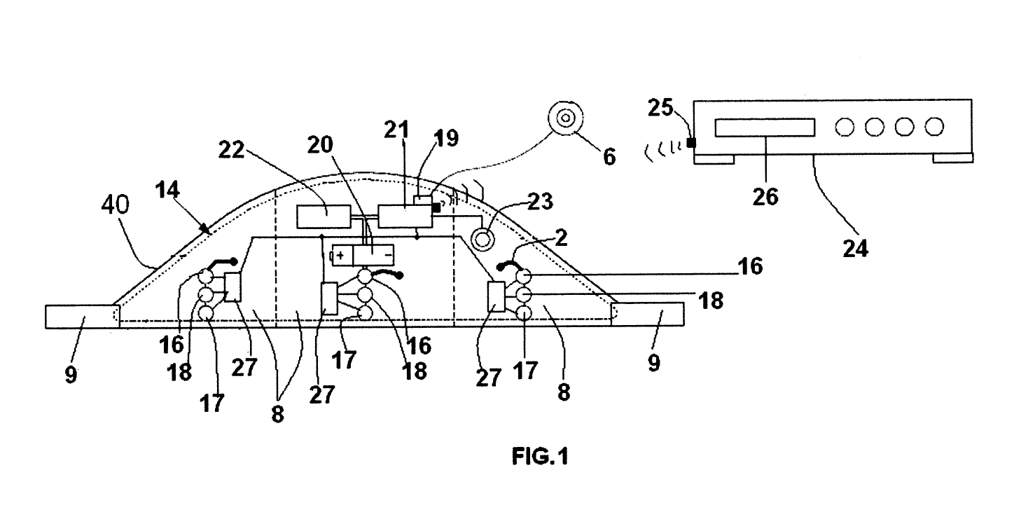

With reference once again to FIG. 1, applied directly on the belt 1 is an electropneumatic apparatus, which comprises, for each inflating chamber 4, at least a microcompressor 16, a pressure sensor 17, and a solenoid valve 18. The belt 1 may moreover be equipped with a battery assembly 20, preferably of a rechargeable type, and a card 21, for example a Bluetooth interface card, as well as USB/wireless/GSM-CPRS communication modules, or any other technological arrangement for wireless data exchange at a distance and/or remotely with an external processing unit 24 provided with a corresponding wireless interface 25 and possibly a display 26 for enabling signalling of values of the parameters and other information useful for handling delivery.

In a possible embodiment, the card 21 will further comprise an electronic circuitry 19 for supply and control and for pre-amplification of the signals coming from a system of sensors 6 for monitoring significant vital parameters of the parturient and/or of the foetus.

Preferably, the battery assembly must be readily accessible so that it can be removed and recharged in a battery charger separate from the belt.

With reference to FIG. 1, the belt 1 may finally be equipped with a display 22 and an acoustic warning device 23, for example, for warning of a malfunctioning of the system (e.g., excess of pressure, batteries run down, faults in data transmission).

In a preferred embodiment, the battery 20 is directly connected to the main circuit of the belt from which there depart the supply and control connections to any possible secondary card 27 that controls operation of the electropneumatic components of the inflating chambers 8 in the respective areas of pressure.

By way of example, there may be envisaged the use of compressors 16 constituted by rotary micropumps (for example, marketed by the firm Okenseiko) supplied at 32 Vdc, which develop a maximum pressure of 0.6 bar, with a flowrate of from 90 to 800 cc/min and a current absorption of from 80 to 640 mA each, to be set according to the size of the chamber 8 to be inflated. The solenoid valves 18 may be mini-solenoid valves that enable deflation in a controlled way of the bladder, for example solenoid microvalves available on the market as KSV05A supplied at 32 Vdc, which absorb 75 mA, and with adequate flows for a rapid deflation (i.e., such as to cause a 100-cc container to pass from 0.4 to 0.02 bar in 32 seconds).

The pressure sensors 17 can finally be miniaturized piezoresistive sensors suited for printed circuits, with adequate measurable pressure intervals, for example integrated sensors of the type available on the market as MS5565 produced by the company Intersema® with electronics for conversion of the data into 16-bit digital format, with resolutions of 0.1 mbar and power consumptions of just 5 μA.

FIG. 8 illustrates a possible embodiment of the electropneumatic apparatus 15 of the type used in digital sphygmomanometers available on the market.

In this solution, the electropneumatic components (compressor 16, valve 18, inflating chamber 8, pressure sensor 17) are connected together, and the air can enter only through the minipump 16 through the inlet duct 2 and exit only through the relief solenoid valve 18.

Advantageously, by controlling operation of just the micropump 16 and of the solenoid valve 18 it is possible to adjust the pressure within the chamber 8, monitoring it constantly with the pressure sensor 17.

In use, the belt 1 can be worn by the parturient, and the sensors 6 can be applied for detecting the significant parameters, according to the particular clinical situation.

By way of example, the sensors 6 may be ECG sensors, tocographic sensors, ultrasound sensors, or sensors of electromyographic activity.

The data detected by the sensors 6 are received by the electronics 21 of the belt and transmitted via the wireless interface to the external processing unit 24 for assessment by the medical staff.

On the basis of the information received, via the unit 24 the medical staff can control operation of the apparatus 15 and in particular the possible inflation or deflation of the chambers 4.

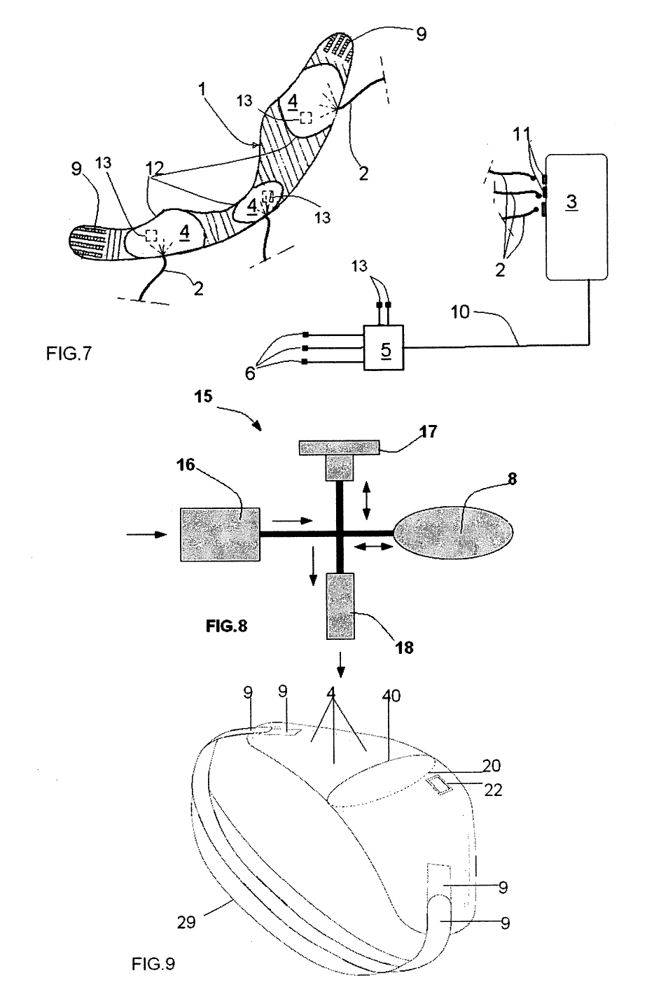

According to a possible aspect of the invention, represented schematically in FIGS. 5 and 7, the belt 1 comprises multiple areas of inflation 4 connected via respective inflation ducts 2, which are in turn connected to the nozzles 11 of an external supply 3 of a pressurized fluid, for example compressed air.

Represented schematically in FIG. 7 is also an apparatus for assistance to delivery, comprising the belt of FIG. 5, a source 3 of pressurized fluid, and an electronic unit 5 for controlling the source 3.

According to the invention, the unit 5 is connected to pressure sensors 13 that measure the pressure in the areas of inflation 4 to enable or disable upon command inflation of said areas in response to the pressure detected.

The unit 5 is moreover connected to one or more biometric sensors 6, which measure the parameters that are significant for the conditions of delivery, and is able to process said values for enabling or disabling inflation of the areas 4 upon command (automatically or with manual intervention on the part of the operator) in response to the values detected by the sensors themselves.

In use of the belt, when the physician and/or the health staff evaluate (via USB/wireless/GSM-GP RS communication modules and any other technological arrangement for communication at a distance and/or remotely) that, on the basis of the parameters monitored via the sensors 6 (in particular the bioelectrical signals), it is necessary to exert a thrust on the abdomen of the parturient, they may control, via USB/wireless/GSM-GPRS communication modules and any other technological arrangement for communication at a distance and/or remotely, the apparatus for inducing in the belt the desired pressure in one or more of the inflating chambers 4 for the time deemed expedient.

In preferred embodiments of the invention, the control of the belt 1 may be managed at a distance and/or remotely by the health staff or else by the parturient herself, who hence may be in hospital, in the delivery room, in the ward, in bed, at home, via commands applied directly on the belt.

When it is desired to intervene in a differentiated way, for example for repositioning the foetus it will be sufficient, by means of the separate tubes 2, to inflate the appropriate areas independently in order to exert differentiated pressures in different points of the abdomen.

In a further preferred embodiment, each belt is equipped with a recognition code, for example an RFID chip or a chip of some other type for automatic recognition by the system, via a purposely provided antenna.

Advantageously, with this solution, if the instrument does not recognize the belt at the moment of startup, the software warns the user and does not enable execution of any operation on the belt.

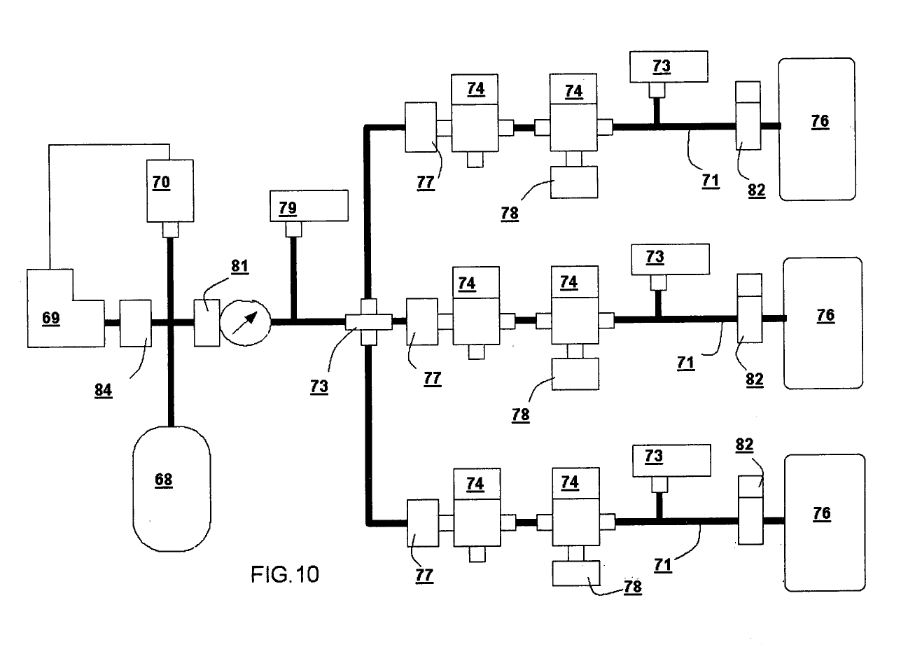

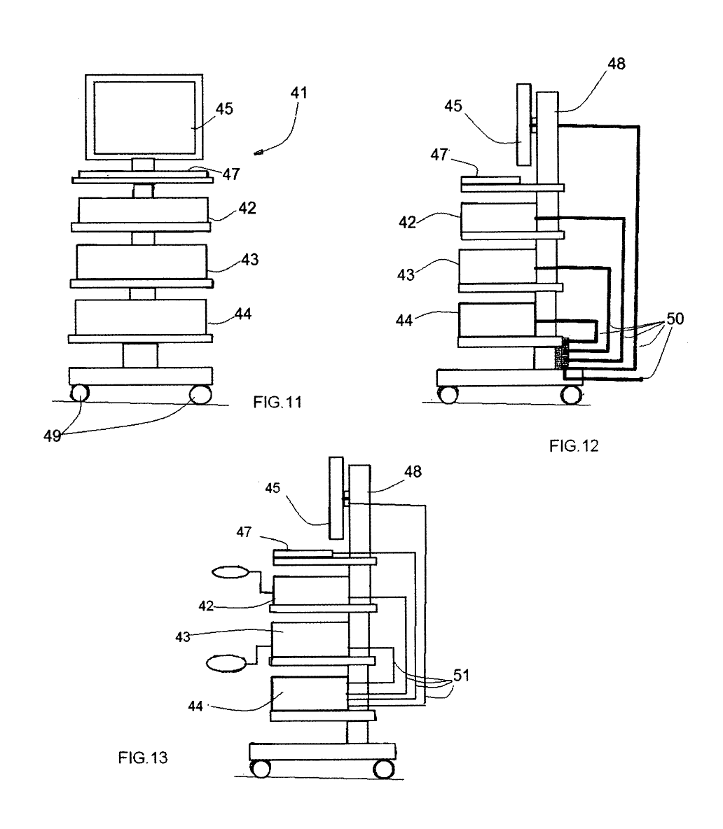

With reference to FIGS. 10-18, an electromedical apparatus 41 according to the invention is described, which is designed for assistance and monitoring of the mother and the foetus during delivery or during labour wherever this may occur.

The apparatus comprises a monitoring device 42 connected to a processing unit 44, for example a personal computer provided with input peripherals 47 such as a keypad and a mouse, and moreover connected to a system for supplying information to the medical and health staff, for example a monitor 45. In the example represented in FIGS. 10-13, the components of the apparatus are supported by a column structure 48, preferably mobile on wheels 49, which carries the electric-power supply cables 50 and the connection cables 51 for communication of data and signals, for example optical-fibre cables, cables for serial and USB connections, and any other type of wireless/GSM-GPRS connection that may be used, as well as any other technological arrangement for communication at a distance and/or remotely.

With reference to FIG. 16, a diagram of a monitoring device 42 according to the invention is illustrated in greater detail.

The device 42 comprises an electronics 56 for acquisition of the signals s1, s2 coming from a plurality of sensors 46, amongst which one or more bioelectrical sensors, for example electrocardiogram (ECG) sensors 52 of the type that can be applied superficially on the abdomen of the mother.

The sensors 52 can in particular be used for monitoring heart rates of the parturient and of the foetus (or foetuses in the case of multiple births), in addition to being used for acquiring electromyographic signals representing the contractions of the parturient.

In addition to the ECG sensors 52, the device 42 is connected via connectors 58 to non-ECG sensors for detecting different vital/biological parameters of the mother and/or of the foetus, such as, for example, one or more tocographic sensors 53, one or more ultrasound sensors 54, and one or more saturometers 55.

It is understood that in the apparatus of the invention sensors of a different type may in any case be used. such as Hotter sensors or others still.

According to the invention, the same acquisition unit 56 receives the signals s2 detected by the non-ECG sensors via an intermediate circuit 57, which conditions said signals and converts them into a format homogeneous with the format of the signals s1 sent by the ECG sensors to the unit 56.

Advantageously, with this solution it is possible to manage in a uniform way and with the same acquisition unit signals that are different from one another, which represent any vital/biological parameter of interest and which can be sent simultaneously to the I/O peripherals of the processing unit 44 for processing with the appropriate software (SW) programs and for subsequent communication or display to the medical and/or health staff.

Preferably, the graphic representation of the parameters acquired may thus be managed in an integrated way, and displayed in an optimized way, for example with the time scale that is most suitable for evaluating the parameters detected or else such as to highlight to the medical and health staff the combination of just the parameters useful for taking decisions in a given circumstance, possibly processed by the unit 4 for supplying a necessary clinical response.

In this connection, it should be recalled that right from the start of labour, whether this be at home or in hospital, spontaneous or else induced by drugs, or even in delivery analgesia, it is important to monitor a series of vital/biological parameters of the mother and foetus, as well as parameters of the uterus.

In particular, it is known that the foetus modifies its own heartbeat depending upon the situation in which it finds itself and upon the state of oxygenation of the heart and brain.

Furthermore, during contraction of the uterus, the pressure of the uterus on the foetus can reveal states of foetal hypoxia through modifications of the foetal heartbeat.

Advantageously, the apparatus of the invention enables an integration between the sensors necessary for detecting evolution of the foetal heart rate and that may possibly be used for carrying out an electrocardiogram on the foetus, and the sensors that monitor the uterine contractions and the heartbeat of the mother (heart rate and/or ECG).

In fact, even in situations such as during delivery analgesia, the invention enables monitoring, not only of what has been described above, but also of the heart of the mother by means of evaluation of the heart rate, or else execution of an electrocardiogram or else monitoring of the percentage of oxygenation of the blood of the mother.

The possibility of offering to the health staff combined information and a video representation (even at a distance and remotely) of all the parameters considered hence enables an overall knowledge of the effective state of health of the mother and foetus and a forecast on the presence or otherwise of delivery risks.

In this connection, it is emphasized that, since the delivery risk is unforeseeable but always present, both in low-risk situations and in higher-risk situations (slight gravidic hypertension, gestational diabetes, previous deliveries with detachment of placenta, dystocia), having available a simultaneous representation of the different significant parameters of both the mother and the foetus, even at a distance and remotely, is very useful for the health staff, also in relation to the fact that it is not possible to foresee the delivery times in advance.

Thanks to the invention, the acquired values of the parameters monitored can be processed for supplying clinical information, which remains traceable.

For example, the knowledge of saturation of the oxygen measured by means of a saturometer is indicative of maternal vital parameters and provides an index of perfusion useful both for monitoring the mother during delivery analgesia and when it is necessary to take urgent decisions or when a closer patient-physician interaction is desirable.

For this purpose, on the screen 45 also at a distance and remotely there may be displayed permanently the traces of the ECG electrodes 52 and of the tocographic probe 54, in addition to the values of the foetal pulsations and of the saturation of the oxygen of the mother, obtained from analysis of the signals coming from the ultrasound probes 53 and from the saturometer 54 (FIG. 16).

Furthermore, the software of the unit 44 may envisage recording of the bioelectrical traces and management of demographic data regarding the patients, and data of labour and delivery, and management of the data on the stem cells in connection with the computerized clinical charts that may be present at the competent health authority.

It is understood, however, that the nature and form of representation of the most significant parameters may differ from case to case and be managed in a targeted way for each particular case, even at a distance and remotely and at the home of the patient.

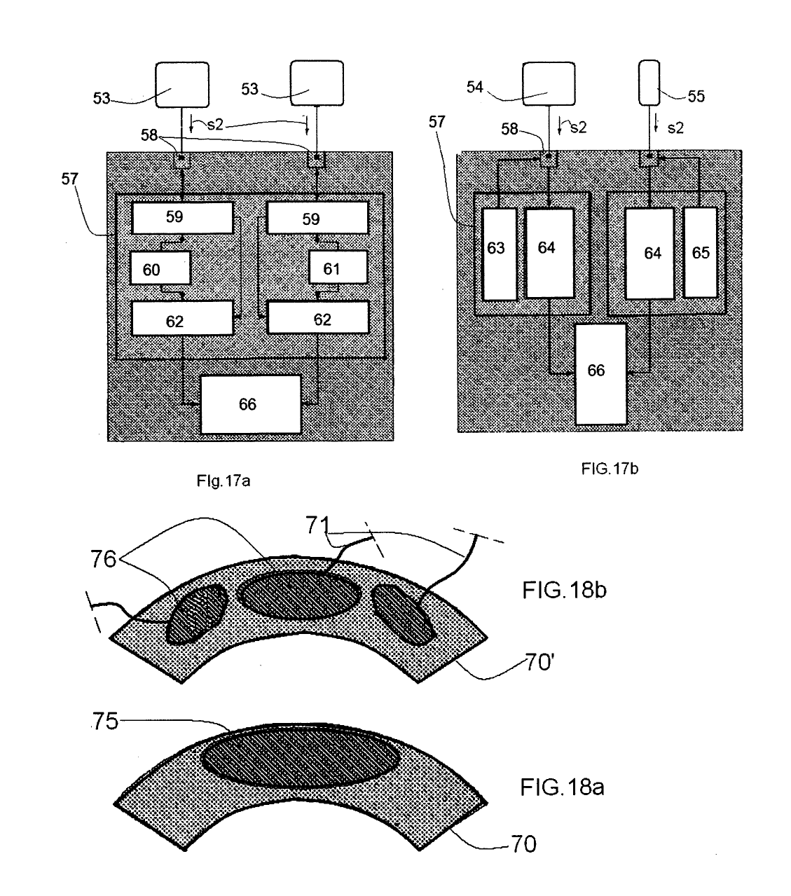

With reference to FIG. 17a, illustrated schematically is an intermediate circuit 57 for pre-processing signals s2 coming from one or more ultrasound probes 53 (in the example described two probes).

In this case, the circuit 57 comprises two transmitter/receiver units 59 associated to a respective sensor or probe 53, two frequency generators 60, 61 at different frequencies, and two signal-combination and filtering units 62, communicating with the electronics 56.

The US probe, via a piezoelectric transducer, alternates in time the function of emission and reception of ultrasounds. In the emission step, the piezoelectric is energized with an electrical signal at a known frequency, generated by the unit 59, typically at around 1-3 MHz. The acoustic signal thus generated penetrates through the abdomen of the mother and is progressively reflected, modified in frequency by the blood.

In the detection step, the piezoelectric converts the frequency of the ultrasound wave reflected into an electrical signal of equal oscillation, received by the unit 59. The signal generated and the one received are combined, filtered, and adapted in amplitude by the unit for combination of the signals 62, so as to isolate just the difference signal, with a frequency of the order of tens of hertz, which is acquired by the electronics 56.

The differences between the frequency sent and the one received indicates the relative flow rate of the blood, and, from the analysis of these values in succession, the heart rate of the foetus and/or the pattern of the blood flow are obtained.

Preferably, in the case of use of two US probes simultaneously, it is necessary for the two frequencies of excitation of the piezoelectric, generated by the generators 60 and 61, to be sufficiently different to enable a spectral distinction between the two signals.

Advantageously, the use of two US probes enables both evaluation of the heart-rate variation of the foetus and Doppler-flowmetric monitoring, which exploits the Doppler effect to detect, through the variations in flow rate of the blood, the pattern of the blood flow in the vessels of the foetal umbilical cord or of other foetal vessels, these being parameters that it is important to monitor to acquire information and be able to take decisions in managing labour and timing of delivery.

Alternatively, using a US probe, an echography could be carried out on the foetus to check, for example, how the foetus presents, the amount of amniotic liquid, the placenta.

With reference to FIG. 17b, a possible intermediate circuit 57 is schematically illustrated for pre-processing signals s2 coming from a tocographic probe 54 of a type in itself known, constituted by a pressure transducer that modifies the voltage at input in proportion to the deformation that it has undergone, and hence to the pressure applied, and a saturometer 55, also this of a type in itself known and made up basically of an appropriate light source and a photodetector in such a way that the light emitted at particular wavelengths, typically red 660 nm and infrared 940 nm, interacts with the subcutaneous blood vessels and is received and converted into electrical signal by the photodetector to supply a signal that depends upon the variations of the oxygenation of the blood, which entail a different absorption of the incident light, and that is used for controlling maternal vital parameters particularly in spontaneous deliveries with delivery analgesia.

In this case, the circuit 57 associated to the tocographic probe 54 possibly comprises a circuit 63 for energizing the tocographic transducer 53, and a circuit 64 for reception of the signals s2 to be sent to the processing unit 16, whilst the circuit 57 associated to the saturometer 55 comprises a supply circuit 65 and in turn a circuit 64 for reception of the signals s2 to be adapted and sent to the processing unit 56.

Advantageously, the adaptation circuits 57 enable acquisition of the signals coming from the tocographic probe 54 and from the saturometer 55, which are electrical signals with values of voltage and current comprised in a range different from the one that the electronics 56 may accept.

The circuit 57 hence makes it possible in this case to modify linearly, via two different circuits 64, the interval of variation of the signals at output from the probes to the interval of acceptability of the signals at input to the electronics 56 for amplification of the signals so that they can be read and processed in a combined and integrated way.

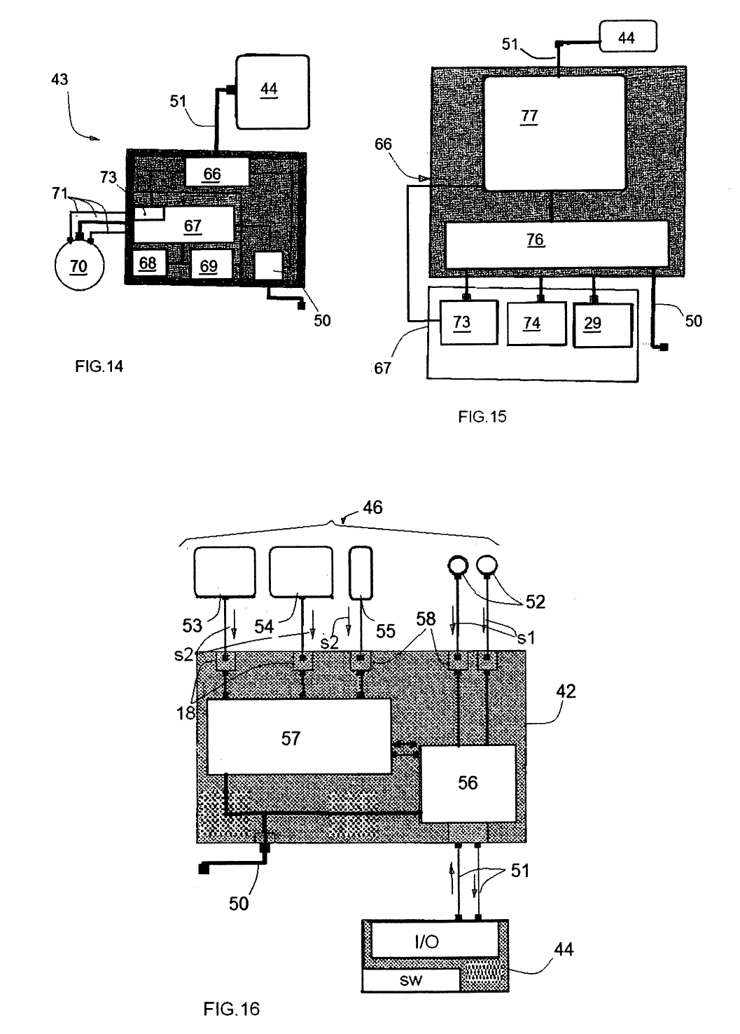

Described with reference to FIGS. 14 and 15 is a possible diagram of an electropneumatic assembly 43 according to the invention.

The assembly 43 comprises:
an inflatable belt 70, made preferably of biocompatible material for application to the abdomen of the parturient;
a pneumatic circuit 67, comprising pressure sensors 73 that measure the pressure of the belt 70, and a compressor 69, connected to the belt 70 by means of one or more tubes for compressed air 71; and
a control electronics 66 connected to the unit 44 and to the sensors 73.

In greater detail (FIG. 15), the control electronics 66 comprises:
a supply circuit 77, which provides the electric-power supply for the sensors 73, one or more solenoid valves 74, and the compressor 69; and
an input/output card 77, connected to the sensors 73, to the supply circuit 66, and to the main electronic unit 44.

Preferably, there may then be provided pressure sensors for measuring the thrust exerted by the abdomen of the parturient on the belt.

In operation, the belt 70 is applied to the abdomen of the parturient, and the sensors 73 are arranged for measuring the pressure of the air in the belt.

The belt 70 is then possibly connected to the compressor 69 via the solenoid valves 74, which enable checking of the steps of pressurization and depressurization of the belt on the basis of the signals supplied by the unit 44 to the control electronics 66 (either automatically or entered by the medical staff) and from this to the compressor and to the valves.

In particular, when the physician and/or the obstetrician even remotely and at a distance evaluate/s that, on the basis of the parameters monitored via the sensors 46 (in particular the bioelectrical signals), it is necessary to exert an action of accompaniment to the contraction or a controlled pressure on the abdomen of the parturient, they/he may introduce via the peripherals 47 of the central unit 44 the data corresponding to the desired effect, and the unit 44 will transfer to the control electronics 66 the commands for inducing in the belt the desired pressure and activating the compressor for inflation of the belt 70 with the intensity and for the time deemed appropriate even remotely and at a distance.

In a preferred embodiment, illustrated in FIGS. 18a and 18b, the belt 70 may comprise a single inflatable chamber 75 or a number of chambers 76, connected to the pneumatic circuit by means of separate tubes 71 that can be inflated independently, and possible further safety valves 82 in order to exert differentiated pressures at different points of the abdomen and thus control, for example, the positioning of the foetus with respect to the neck of the uterus during labour or prior to expulsion.

In a further preferred embodiment, each belt 70 is equipped with an RFID chip or a chip of some other type for automatic recognition by the system, via a purposely provided antenna.

Advantageously, with this solution, if the instrumentation does not recognize the belt at the moment of start-up, the software warns the user and does not enable any operation to be carried out on the belt.

With the apparatus described it is moreover possible for the record of each use of the instrumentation, with the values of the pressures detected and the manoeuvres performed, to be stored in an appropriate digital format and, together with the demographic information on the patient and the monitoring traces, to come to constitute a complete digital file of all the values monitored and the procedures carried out during each delivery. Managed locally, or else remotely in the case where the system is network-connected, the record constitutes a sort of labour/delivery black box, whether labour/delivery has taken place at the home of the patient or in hospital.

From the above description, it is evident that with the apparatus of the invention it is possible to assist the uterus in the contractions during labour and replace the Kristeller manoeuvre, preventing the adverse consequences thereof through a specific clinical monitoring that will provide the health operators also at a distance and remotely with an instrument for controlling the uterine contractions and the other significant parameters.

In fact, thanks to the monitoring device, via the use of different sensors, the traces of the signals detected are displayed on the monitor even at a distance and remotely so that the physician and/or the obstetrician are/is put in a condition where they are able to assess how the labour and/or delivery are/is proceeding, through visual analysis of the heart rate and heart-rate variation of the mother and of the foetus and of the uterine contractions, and of the other relevant parameters case by case.

Furthermore, thanks to the pressure device integrated in one and the same apparatus, in the case where the physician and/or the obstetrician, on the basis of the traces and of the values displayed on the monitor, deem/deems that the contractions need to be accompanied by pressure and that the foetus and the parturient have good heart parameters, they/she can activate the pressure device, even at a distance and remotely, and accompany and/or induce a series of uterine contractions in order to stimulate natural delivery.

Advantageously, an insulation transformer in compliance with current standards guarantees the electrical safety of the system, and the parts applied of the monitoring system form part of a medical device in conformance with the safety directives.

Furthermore, the part of the pressure instrument applied to the abdomen of the parturient does not present elements of electrical conduction and is basically made up of a belt of biocompatible material, with one or more air chambers inside it, which can be inflated through one or more tubes of insulating plastic material.

The energy applied to the patient through the belt is hence exclusively of a pneumatic type for impressing on the abdomen of the parturient a pressure that indicatively may be, for example, between 60 mmHg, and 200 mmHg, equivalent to 266 mbar, without causing problems of any kind.

Preferably, as pressure generator a compressor of small dimensions is used, capable of delivering a maximum pressure on the belt, equal to a value that is not risky for the parturient and is indicatively approximately 0.25 bar. The pressure device comprises a small reservoir 68 and a manual pressure regulator 81, which, appropriately set during testing, enables a regular air flow without any peaks. Finally, the pressure on the belt is monitored by a first high-sensitivity pressure sensor 73, preferably with a range of between 0 and 1 bar, positioned in the circuit for supplying compressed air to the belt 70.

The pressure on the belt is regulated preferably by the action of two solenoid valves 74 set cascaded, the open-closed combinations of which enable inflation and deflation of the belt, whenever required, and maintenance of the desired pressure reached.

The two solenoid valves are preferably of the normally closed type, in such a way that at rest, i.e., without supply, they do not allow the compressed air upstream to reach the belt downstream and, at the same time, enable the belt to deflate in the case of anomalous events.

In the absence of electrical supply, the pressure circuit of the belt 70 is open and at the moment of turning-off, whether voluntary or accidental, it assumes a configuration such as to deflate the belt.

A second pressure sensor 73 is set upstream of the solenoid valves 74 and monitors the pressure at output from the compressor 69 and is useful as further control in the steps of diagnostics in start-up and during normal operation. Consequently, the control on the pressure exerted is such as not to allow risky manoeuvres on the patient even in conditions of a number of simultaneous failures.

In particular in the worst condition of the compressor 69 being blocked whilst still turned on, simultaneously with the occurrence of jamming of the valves 74 in a position such as to cause inflation of the belt, and of malfunctioning of pressure switch 80, the maximum pressure that the belt can exert would be in any case lower than the value that has already been found to be harmless.

In the final analysis, as last safety device, it is possible to provide an arrest pushbutton, set in a position that is readily accessible to the operator, which enables the operator to intervene by acting directly and simultaneously on the solenoid valves 74, as electrical switch, without passing through the control electronics, obtaining the immediate depressurization of the belt, independently of the operation that the system is currently performing.

The invention now described affords important advantages in monitoring of the most significant steps of labour and delivery, and provides the health staff with an integrated instrument, which can be used even at a distance and remotely, in the delivery room, at the patient's bedside, in the ward or at home, in the different situations that can occur or evolve in management of labour and delivery and where it is necessary or useful to be able to carry out a monitoring of the vital and/or biological parameters with technically and functionally distinct sensors, having available a clinical instrument for processing signals, either singly or taken in combination.

In a preferred embodiment of the invention, the combined use of an active system is envisaged for exerting a controlled pressure for accompanying the contractions of the uterus during labour and/or delivery.

Advantageously, the integration of the pressure system in the same monitoring apparatus controlled by one and the same processing unit enables ,the health staff to decide how, whether, and when to intervene on the basis of the parameters observed, or to modify the clinical observation of the parameters to be able to take different decisions.

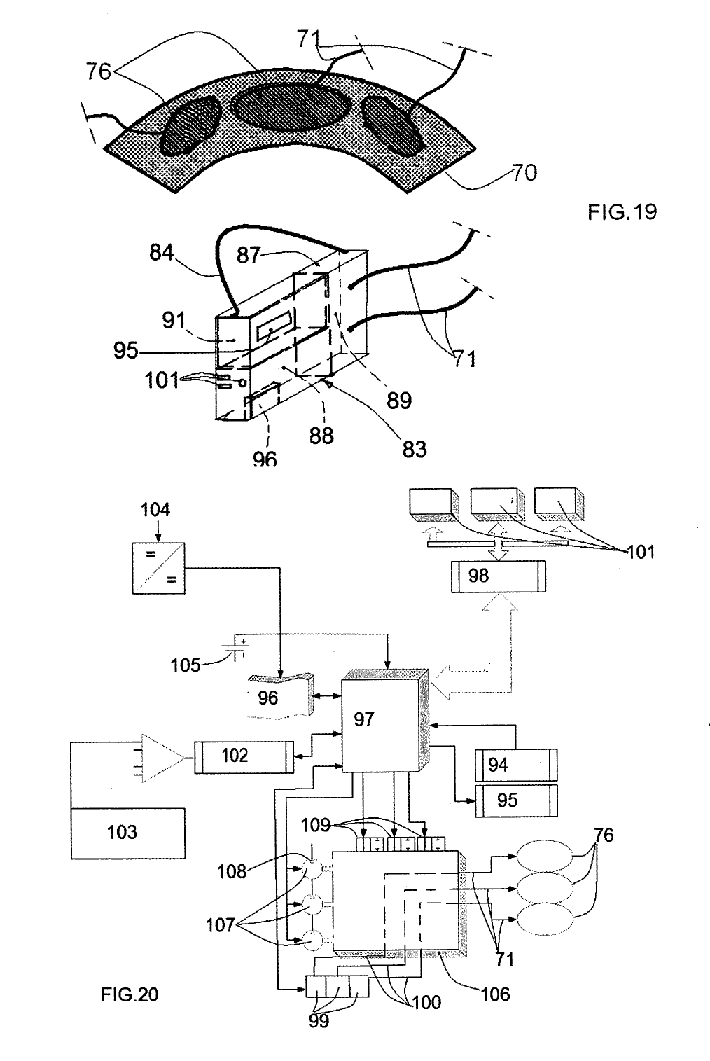

FIG. 19 illustrates a preferred embodiment of an apparatus according to the invention.

Also in this embodiment, the specific technical purpose is to provide the parturient with the maximum possible mobility.

The solution devised envisages that the apparatus comprises a portable device 83, provided for example with a shoulder strap 84, with which it is possible to accompany the parturient from hospitalization up to entry into the delivery room, or else conveniently at home, in bed or when the patient is moving around, under the control of the obstetrician, and with the possibility of being checked also at a distance and remotely, the USB/wireless/GSM-GPRS communication modules maintaining a monitoring of the parameters up to delivery.

Finally, a further characteristic is to create a thorough-going traceability, not only of the actions of active aid, via an inflatable band, but also of the physiological parameters of the mother.

Advantageously, the components used have been miniaturized so as to provide the possibility of rendering the portable device 83 battery-operated and able to supervise all the functions without the need to connect it physically to an external PC for its use.

The device 83 is constituted by a container 87 that contains an electronic section 88 and a pneumatic section 89.

The pneumatic section 89 may be connected by means of three tubes 71 to one and the same inflatable band 70 to be applied on the abdomen of the mother, for example of the type described previously.

The portable device 83 may moreover be equipped with a battery 91 for mobile use and with connections 101 for example of a USB type (Master and Slave) and with a wireless connection, for example Bluetooth®, to be used as channel for data exchange with a remote server or else for USB/wireless/GSM-GPRS communication.

According to the invention, it will be in any case be possible to connect the device 83 to an external power supply that will serve either as direct power supply or as battery charger.

In greater detail, with reference to FIG. 20, the electronic section 88 preferably comprises:
commands 94 for regulating and controlling the inflating pressures of the chambers 76;
a display 95 for displaying the data entered and read in real time;
a storage unit 96 for acquisition of the operating data entered and of the read data; preferably the memory 96 is of a non-erasable type and enables acquisition of a series of data and association thereof to a time scale so as to create a thorough-going “black box”;
a microprocessor computing unit 97;
units 98 for managing the I/O peripherals; and
sensors 99 for measuring the pressures.

Preferably, the sensors 99 are pressure transducers housed in the electronic section 88 and in fluid communication with pipes 100 coming from the pneumatic unit and communicating in turn with the chambers 76 via the tubes 71, a unit 102 for acquisition of the ECG biomedical signals of the foetus and of the mother, uterine contractions, a Doppler and tocographic acquisition unit 103, and a low-voltage supply unit 104, preferably pre-arranged for connection with a battery-charger device.

From the functional standpoint, the pneumatic section 84 can be substantially of the type illustrated in FIG. 10 and comprises in particular one or more (for example three) independent pneumatic circuits each made up of a rotary-pump microcompressor 107, a non-return valve 108, a solenoid valve 109 for inflation of the chambers 76, and a fast-block coupling for distribution of the air towards the inflatable belt 70.

In a preferred embodiment, all three circuits are arranged within a miniaturized enbloc assembly 106 made of aluminium or other suitable material, within which the pneumatic layout is implemented.

The present invention has been described according to preferred embodiments, but equivalent variants may be devised without departing from the sphere of protection granted.

1. An inflatable belt as aid to labour and/or delivery, comprising a supporting band and at least one inflatable chamber applied to said band and provided with ducts for connection to a supply of an inflating fluid, said belt comprising an electropneumatic apparatus applied on the band and, for each inflating chamber, at least one microcompressor for inflation of the chamber, at least one pressure sensor for measuring the pressure reached in the chamber, and at least one solenoid valve for discharging the pressure.

2. The belt according to claim 1, comprising at least one electronic unit for controlling said electropneumatic apparatus, connected with a sensor system for detecting vital/biological parameters of the parturient and/or of the foetus and provided with a wireless interface for exchange, with an external processing unit, of signals correlated to said parameters, even remotely and/or at a distance.

3. The belt according to claim 1, comprising a secondary electronic card, connected to said main electronics for controlling the electropneumatic apparatus and associated to each inflating chamber.

4. The belt according to claim 1, comprising at least two areas of inflation that can be activated individually via respective inflation ducts.

5. The belt according to claim 1, wherein said areas of inflation are set along a longitudinal axis of wrapping of the belt around the abdomen.

6. The belt according to claim 1, wherein said areas of inflation are set transversely with respect to a longitudinal axis of wrapping of the belt around the abdomen.

7. The belt according to claim 1, wherein said inflating chambers are separate sectors of a single inflating chamber.

8. The belt according to claim 1, further comprising unique identification code associated thereto.

9. The belt according to claim 1, further comprising a battery.

10. An apparatus for assistance to childbirth, comprising one or more belts according to claim 1 and a processing unit connected in wireless mode to the electronic unit of said belts, said apparatus being usable at a distance and remotely.
